(19) United States
(12) Patent Application Publication
(10) Pub. No.: US 2025/0188490 A1
(43) Pub. Date: Jun. 12, 2025

(54) METHODS OF RAAV PACKAGING

(71) Applicant: University of Massachusetts, Boston, MA (US)

(72) Inventors: Dan Wang, Worcester, MA (US); Hao Liu, Worcester, MA (US); Yue Zhang, Worcester, MA (US); Guangping Gao, Worcester, MA (US)

(73) Assignee: University of Massachusetts, Boston, MA (US)

(21) Appl. No.: 18/844,502

(22) PCT Filed: Mar. 8, 2023
(86) PCT No.: PCT/US2023/063977

§ 371 Date: Sep. 6, 2024

Related U.S. Application Data
(60) Provisional application No. 63/317,933, filed on Mar. 8, 2022.

(51) Int. Cl. C12N 15/86

(52) U.S. Cl. CPC C12N 15/86; C12N 2710/10044; C12N 2750/14122; C12N 2750/14143; C12N 2750/14152

(57) ABSTRACT

In some aspects, the disclosure relates to methods for improving titer and yield of viral vector production. In some embodiments, the disclosure relates to compositions and methods of using same, wherein the compositions comprise (i) a cis-element nucleic acid comprising a transgene: (ii) a helper nucleic acid encoding adenoviral helper genes; and (iii) a packaging nucleic acid encoding Rep and/or Cap genes: wherein the ratio of (i): (ii) and/or the ratio of (i): (iii) is between 0.01:1 and 0.1:1.

### RELATED APPLICATIONS

This application is a national stage filing under 35 U.S.C. § 371 of international PCT application PCT/US2023/063977, filed Mar. 8, 2023, which claims priority under 35 U.S.C. § 119 (e) to U.S. provisional patent application, U.S. Ser. No. 63/317,933, filed Mar. 8, 2022, the entire contents of each of which are incorporated by reference herein.

### BACKGROUND OF INVENTION

Recombinant adeno-associated virus (rAAV) vectors are the leading platform for human gene therapy delivery. In addition, rAAV technology is widely used for functional genomic studies in biomedical research, such as molecular genetics and cancer biology. Accordingly, robust production of high-titer rAAV packaging of a desired transgene is necessary for clinical applications and basic research.

### SUMMARY OF INVENTION

Aspects of the disclosure relate to methods of packaging rAAV vectors (e.g., cost-effective and universal methods of packaging rAAV vectors with non-cytotoxic or cytotoxic transgenes). The inventors discovered that substantially reducing the cis-element plasmid input (e.g., by 10-fold to 100-fold, relative to other input plasmids) in a triple transfection protocol did not compromise the availability of rAAV genome or rAAV production. Furthermore, the inventors found that this substantial reduction in cis-element plasmid input did not negatively impact rAAV yield. Methods of the disclosure maintain high yield of rAAV production and enable efficient packaging of cytotoxic transgenes while reducing the monetary costs of rAAV packaging (e.g., by reducing the amount of cis-element plasmid by up to 99%).

In some aspects, the disclosure provides a method for producing recombinant adeno-associated virus (rAAV). In some embodiments, a method for producing rAAV comprises introducing into a host cell: (i) a cis-element nucleic acid comprising a transgene; (ii) a helper nucleic acid encoding adenoviral helper genes; and (iii) a packaging nucleic acid encoding Rep and/or Cap genes; wherein the concentration of the cis-element nucleic acid that is introduced to the host cell is 10-fold to 100-fold less than the concentration of the helper nucleic acid and/or the packaging nucleic acid that is introduced to the host cell.

In some embodiments, a method for producing rAAV comprises introducing into a host cell: (i) a specific concentration of cis-element nucleic acid comprising a transgene; (ii) a specific concentration of helper nucleic acid encoding adenoviral helper genes; and (iii) a specific concentration of packaging nucleic acid encoding Rep and/or Cap genes; wherein the ratio of (i): (ii) and/or the ratio of (i): (iii) is between 0.01:1 and 0.1:1.

In some embodiments, the host cell is a viral vector packaging cell. In some embodiments, the host cell is a mammalian cell. In some embodiments, the host cell is a human cell, optionally a HEK 293T cell. In some embodiments, the host cell is an insect cell, optionally a Spodoptera frugiperda (Sf9) cell.

In some embodiments, the cis-element nucleic acid, the helper nucleic acid, and/or the packaging nucleic acid is a plasmid. In some embodiments, the cis-element nucleic acid comprises a transgene flanked by two inverted terminal repeats (ITRs). In some embodiments, at least one of the ITRs is an AAV1, AAV2, AAV3, AAV4, AAV5, AAV6, AAV7, AAV8, or AAV9 ITR. In some embodiments, the cis-element nucleic acid is a self-complementary nucleic acid comprising at least one AITR or mTR. In some embodiments, the transgene encodes a protein, optionally a therapeutic protein. In some embodiments, the transgene is cytotoxic or comprises one or more physiochemical characteristics that are detrimental to the fitness of the host cell (e.g., the transgene encodes a protein that forms a secondary structure with high thermal stabilities).

In some embodiments, the adenoviral helper genes comprise E4, E2a and/or VA genes.

In some embodiments, the concentration of the cis-element nucleic acid that is introduced to the host cell is 10-fold to 100-fold, 10-fold to 90-fold, 10-fold to 80-fold, 10-fold to 70-fold, 10-fold to 60-fold, 10-fold to 50-fold, 10-fold to 40-fold, 10-fold to 30-fold, or 10-fold to 20-fold less than the concentration of the helper nucleic acid and/or the packaging nucleic acid that is introduced to the host cell. In some embodiments, the ratio of the concentration of (i) relative to (ii) is between 0.01:1 and 0.1:1, optionally between 0.01:1 and 0.05:1 or between 0.01:1 and 0.025:1. In some embodiments, the ratio of the concentration of (i) relative to (iii) is between 0.01:1 and 0.1:1, optionally between 0.01:1 and 0.05:1 or between 0.01:1 and 0.025:1.

In some embodiments, the cis-element nucleic acid, the helper nucleic acid, and/or the packaging nucleic acid are introduced into the host cell simultaneously. In some embodiments, a composition comprising the cis-element nucleic acid, the helper nucleic acid, and/or the packaging nucleic acid is introduced into the host cell.

In some embodiments, the cis-element nucleic acid, the helper nucleic acid, and/or the packaging nucleic acid are introduced into the host cell separately.

In some embodiments, the helper nucleic acid, and/or the packaging nucleic acid are introduced into the host cell using electroporation or transfection techniques.

In some embodiments, the methods of the disclosure produce rAAVs having a higher purity compared to rAA Vs produced by a conventional transfection method. In some embodiments, the methods of the disclosure produce populations of rAAV, wherein at least 90%, 95%, 96%, 97%, 98%, or 99% of the rAAVs comprise the transgene and do not comprises the plasmid backbones of the cis-element, helper, or packaging nucleic acids. In some embodiments, the methods of the disclosure produce populations of rAAV, wherein fewer than at least 10%, 5%, 4%, 3%, 2%, or 1% of the rAAVs comprise comprise read-through rAAV or reverse packaging rAAV.

In some embodiments, the method produces a lower relative amount of read-through rAAV or reverse packaging rAAV compared to a traditional triple-transfection method, optionally wherein the traditional triple-transfection method produces rAAVs using a 1:1 ratio for (i): (ii) and/or (i): (iii). In some embodiments, the amount of read-through rAAV and reverse packaging rAAV is determined by digital droplet PCR.

In some aspects, the disclosure provides a composition comprising: (i) a cis-element nucleic acid comprising a transgene; (ii) a helper nucleic acid encoding adenoviral helper genes; and (iii) a packaging nucleic acid encoding Rep and/or Cap genes; wherein the ratio of (i): (ii) and/or the ratio of (i): (iii) is between 0.01:1 and 0.1:1.

In some embodiments, the cis-element nucleic acid, the helper nucleic acid, and/or the packaging nucleic acid is a plasmid. In some embodiments, the cis-element nucleic acid comprises a transgene flanked by two inverted terminal repeats (ITRs). In some embodiments, at least one of the ITRs is an AAV1, AAV2, AAV3, AAV4, AAV5, AAV6, AAV7, AAV8, or AAV9 ITR. In some embodiments, the cis-element nucleic acid is a self-complementary nucleic acid comprising at least one AITR or mTR.

In some embodiments, the transgene encodes a protein, optionally a therapeutic protein. In some embodiments, the transgene is cytotoxic or comprises one or more physiochemical characteristics that are detrimental to the fitness of the host cell (e.g., the transgene encodes a protein that forms a secondary structure with high thermal stabilities). In some embodiments, the adenoviral helper genes comprise E4, E2a and/or VA genes.

In some embodiments, the concentration of the cis-element nucleic acid is 10-fold to 100-fold, 10-fold to 90-fold, 10-fold to 80-fold, 10-fold to 70-fold, 10-fold to 60-fold, 10-fold to 50-fold, 10-fold to 40-fold, 10-fold to 30-fold, or 10-fold to 20-fold less than the concentration of the helper nucleic acid and/or the packaging nucleic. In some embodiments, the ratio of the concentration of (i) relative to (ii) is between 0.01:1 and 0.1:1, optionally between 0.01:1 and 0.05:1 or between 0.01:1 and 0.025:1. In some embodiments, the ratio of the concentration of (i) relative to (iii) is between 0.01:1 and 0.1:1, optionally between 0.01:1 and 0.05:1 or between 0.01:1 and 0.025:1.

### BRIEF DESCRIPTION OF DRAWINGS

FIGS. 1A-1C provide a comparison between traditional triple transfection methods and exemplary transfection methods of the disclosure. FIGS. 1A-1B provide an overview of the benefits of utilizing the low-cis element transfection method disclosed herein compared to traditional triple transfection methods. FIG. 1C provides a graph showing that reducing the concentration of cis-element nucleic acids produces comparable titers of rAAV encoding a non-toxic transgene (eGFP) in HEK293 cells (compare lanes 2 and 3 to lane 1).

FIGS. 2A-2C show that reducing the concentration of cis-element nucleic acids enables the expression of rAAV encoding a cytotoxic transgene, which is not compatible with traditional triple transfection methods. FIG. 2A provides a graph showing that the yield of rAAV encoding a suppressor tRNA (stRNA19 or stRNA21) was increased by more than 10-fold when reducing the cis-element amount to 10% or 1% (ratio of 0.1:1:1 cis-element: packaging: helper and 0.01:1:1 cis-element: packaging: helper, respectively). FIG. 2B provides images of western blot experiments showing that the expression levels of Rep and Cap (VP1, VP2, VP3) proteins were increased when reducing the cis-element amount to 10% or 1%. FIG. 2C provides a graph showing that the yield of rAAV encoding a human FOXG1 transgene was increased when reducing the cis-element amount to 10% or 1% (ratio of 0.1:1:1 cis-element: packaging: helper and 0.01:1:1 cis-element: packaging: helper, respectively). 5 FIGS. 3A-3D demonstrate that low-cis element triple transfection methods of the disclosure are capable of packaging cytotoxic UGA sup-tRNA that is otherwise incompatible with standard triple transfection. The packaged rAAV encoding UGA sup-tRNA is functional in rescuing UGA premature stop codon in HEK293 cells. FIG. 3A provides a graph showing that the yield of rAAV encoding mCherry-tagged suppressor tRNA (sup-tRNA19 or sup-10 tRNA21) was significantly increased when reducing the cis-element amount to 10% or 1% (ratio of 0.1:1:1 cis-element: packaging: helper and 0.01:1:1 cis-element: packaging: helper, respectively). FIGS. 3B-3C show that the suppressor tRNAs delivered to HEK293 cells are functional and their delivery is significantly enhanced when reducing the cis-element amount during rAAV production to 10% or 1%. FIG. 3D shows that reducing the amount of transfected pCis maintains functional levels of rep/cap expression, which are critical components involved in rAAV packaging.

FIGS. 4A-4J show that: (1) low-cis triple transfection methods of the disclosure are applicable to both single-stranded and self-complementary vector genomes, various reporter transgenes and serotypes, and (2) this feature is unique to the cis-element plasmid, as reducing pAd-helper or pTrans plasmid input causes dramatic rAAV yield drop. FIGS. 4A-4F demonstrate the rAAV titer of non-cytotoxic transgenes packaged in rAAVs of differing serotypes (AAV2, AAV5 and AAV9) using varying amounts of cis element plasmid. FIGS. 4G-4J show the rAAV titer of non-cytotoxic transgenes packaged in rAAVs of serotype AAV2 or AAV9 using various amounts of pAd-helper (pAF6) or pTrans (pAAV2/9) plasmid.

FIGS. 5A-5H show the applicability of low-cis triple transfection methods of the disclosure in large-scale rAAV production for use in vivo. FIG. 5A shows a graph demonstrating the comparable amounts of scAAV9.CB6.PI.cGFP produced when using 100%, 10% or 1% cis-element plasmid in HEK293 cells. FIG. 5B shows a schematic of the workflow of the mouse study, wherein 8-week-old C57male mice were transduced with empty capsid (control), or with rAAV9 generated using 100%, 10% or 1% cis-element encoding scAAV9.CB6.PI.cGFP. 5 weeks following transduction, the mice were harvested for tissue. FIG. 5C-5D show graphs of the quantification of cGFP DNA (FIG. 5C) and RNA (FIG. 5D) levels in the liver, hearts and muscle (TA) of the mice 5 weeks after rAAV9 transduction. FIG. 5E show quantification of eGFP expression in the livers and hearts of mice transduced with scAAV9.CB6.PI.cGFP. FIGS. 5F-5H show quantification of the expression of protein encoded by the transgene (cGFP protein) in livers (FIG. 5F), hearts (FIG. 5G) and muscle (FIG. 5H).

FIGS. 6A-6E show the applicability of low-cis triple transfection methods of the disclosure for use with multiple transgenes and serotypes for expression of transgenes in vivo. FIG. 6A shows a graph demonstrating the amounts of AAV5.Gluc.BiP.cGFP produced when using 100%, 10% or 1% cis-element plasmid in HEK293 cells. FIG. 6B shows a schematic of the workflow of the mouse study, wherein 8-week-old C57 male mice were transduced with empty capsid (control), or with rAAV5.Gluc.BiP.cGFP generated using 100%, 10% or 1% cis-element encoding AAV5.Gluc.BiP.cGFP. Blood was collected 1 week prior to IV injection with rAAV5.Gluc.BiP.cGFP, then at time points of 3 days, 1 week, 3 weeks and 5 weeks; the mice were harvested for tissue 5 weeks following transduction. FIG. 6C shows a graph of measured Gluc activity (transgene activity) in the serum of mice, with a significant increase in serum Gluc activity in 1% cis mice compared to 100% cis mice. FIG. 6D shows graphs of the quantification of cGFP RNA levels in the liver, hearts and muscle (TA) of the mice 5 weeks after rAAV5.Gluc.BiP.cGFP transduction. FIG. 6E shows quantification of the expression of protein encoded by the transgene (cGFP protein) in the livers and hearts of mice transduced with rAAV5.Gluc. BiP.cGFP.

FIGS. 7A-7E show the superior in vivo transduction of low-cis triple transfection-generated rAAV in liver tissue. FIG. 7A shows a graph demonstrating the amounts of AAV9.Gluc.BiP.cGFP produced when using 100% or 1% cis-element plasmid in HEK293 cells. FIG. 7B shows a schematic of the workflow of the mouse study, wherein 8-week-old C57 male mice were transduced with empty capsid (control), or with rAAV9.Gluc.BiP.cGFP generated using 100% or 1% cis-element encoding rAAV9.Gluc.BiP.cGFP. Blood was collected 1 week prior to IV injection with rAAV9.Gluc.BiP.cGFP, then at time points of 1 week, 3 weeks and 5 weeks; the mice were harvested for tissue 5 weeks following transduction. FIG. 7C shows a graph of measured Gluc activity (transgene activity) in 1% cis mice compared to 100% cis mice, demonstrating the increased GFP fluorescence in livers from mice transduced with 1% cis rAAV9.Gluc.BiP.eGFP. FIG. 7D shows graphs of the quantification of EGFP RNA levels in the liver, hearts and muscle (TA) of the mice 5 weeks after rAAV9.Gluc.BiP.eGFP transduction, with enhanced GFP mRNA expression in the livers of 1% cis mice. FIG. 7E shows quantification of the expression of protein encoded by the transegene (eGFP protein) in livers, hearts and muscles (TA) of mice transduced with 100% or 1% cis rAAV9.Gluc.BiP.eGFP, further demonstrating the superior transduction efficiency of low-cis triple transfection-generated rAAV9.

FIGS. 8A-8F show DNA analyses demonstrating that low-cis triple transfection significantly reduces vector impurity and reverse-packaged rAAV in different serotypes and transgenes. FIG. 8A depicts a schematic of the ddPCR design to detect rAAV with read-through and reverse packaging. FIG. 8B demonstrates the results of ddPCR sequencing with the different probes, showing the enhanced purity of the 10% and 1% cis rAAVs. FIG. 8C shows a denaturing alkaline gel, wherein the arrow points to the oversized read-through band present in the 100% cis rAAV genome but not 10% or 1% cis. FIG. 8D shows quantification of PacBio sequencing of the different regions of the rAAV genome, demonstrating the increased amount of reads mapped to the backbone in the 100% cis rAAV genome but not 10% or 1% cis. FIGS. 8E-8G show further ddPCR results from rAAVs of different serotypes encoding different transgenes, all of which demonstrate the enhanced purity when using 10% or 1% cis compared to 100% cis-elements.

### DETAILED DESCRIPTION OF INVENTION

Traditional triple transfection methods of producing recombinant adeno-associated virus (rAAV), host cells (e.g., HEK293 cells) are co-transfected at a 1:1:1 weight ratio with three plasmids (a helper plasmid encoding adenoviral helper (Ad-helper) genes, a packaging plasmid encoding AAV rep and cap genes, and a cis-element plasmid that encodes a transgene). The transgene may be flanked by ITRs. After triple transfection, the Ad-helper genes drive the expression of rep and cap genes that encode Rep proteins responsible for rAAV genome replication and encapsulation, and Cap proteins-VP1, VP2 and VP3—that form an rAAV capsid.

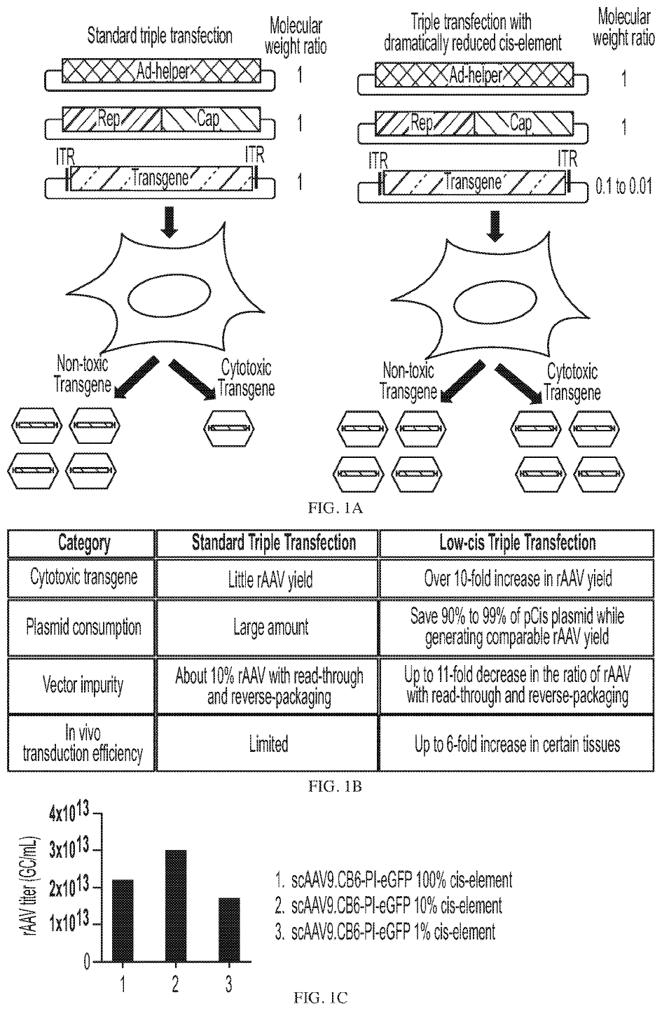

The inventors found that substantially reducing the cis-element nucleic acid input (e.g., by 10-fold to 100-fold, relative to other input nucleic acids, e.g., packaging nucleic acids and/or helper nucleic acids) does not negatively compromise the availability of recombinant adeno-associated virus (rAAV) genome or rAAV production. Thus, triple transfection methods for producing rAAVs with reduced cis-element nucleic acids (e.g., reduction of cis-element nucleic acids relative to other input nucleic acids by 10-fold to 100-fold maintain or provide high yields of rAAV production. In some embodiments, the weight ratio of the cis-element nucleic acid relative to a packaging nucleic acid in a method of the disclosure is between 0.01:1 and 0.1:1. In some embodiments, the weight ratio of the cis-element nucleic acid relative to a helper nucleic acid in a method of the disclosure is between 0.01:1 and 0.1:1. Furthermore, a reduction in the cis-element nucleic acids significantly reduces the cost of rAAV production, by reducing the total amount of the cis-element nucleic acid (e.g., by up to 99% relative to traditional methods). In addition, when transgene expression during rAAV production interferes with the process of rAAV production (e.g., because the transgene is cytotoxic to the host cell(s)), dramatically reducing the amount of cis-element nucleic acid reduces, in some embodiments, transgene expression to a tolerable level such that the host cell(s) can alleviate the interference and generate robust rAAV yield (FIG. 1).

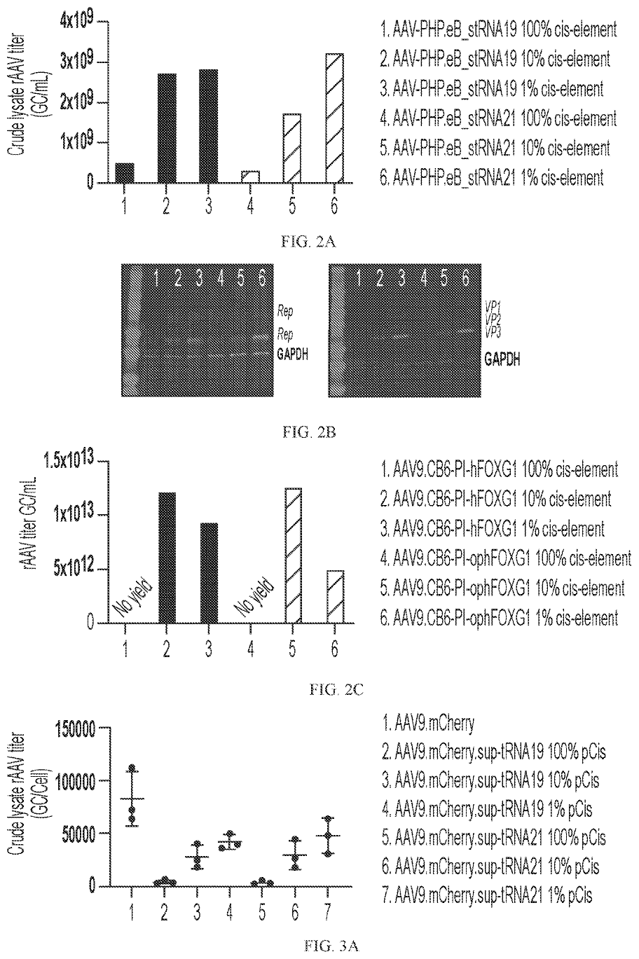

In some embodiments, methods of the disclosure (e.g., triple transfection methods of the disclosure) utilize dramatically reduced cis-element nucleic acid (e.g., cis-element plasmid) encoding transgenes (e.g., non-toxic transgenes such as green fluorescent protein (GFP)). In some embodiments, such methods generates rAAV yields that are comparable to traditional triple transfection methods. For example, in some embodiments, methods of the disclosure (e.g., triple transfection methods of the disclosure) utilize reduced cis-element nucleic acid (e.g., cis-element plasmid), wherein the amount or concentration of the cis-element nucleic acid is reduced by 10-fold to 100-fold relative to the amount or concentrations of the other input nucleic acids (e.g., packaging nucleic acids and/or helper nucleic acids). Such methods of the disclosure that utilize reduced amounts or concentrations of the cis-element nucleic acid generates comparable rAAV yields as traditional triple transfection (See, e.g., FIG. 2).

In some embodiments, methods of the disclosure (e.g., triple transfection methods of the disclosure) utilize dramatically reduced cis-element nucleic acid (e.g., cis-element plasmid) encoding cytotoxic transgenes (e.g., suppressor tRNAs such as UGA suppressor tRNAs) enable production of rAAV titers at elevated levels relative to traditional triple transfection methods. For example, methods of the disclosure that utilize an amount or concentration of the cis-element nucleic acid that is reduced by 10-fold to 100-fold relative to the amount or concentrations of the other input nucleic acids (e.g., packaging nucleic acids and/or helper nucleic acids) can enable production of rAAV titers at elevated levels relative to traditional triple transfection methods. In some embodiments, methods of the disclosure that utilize reduced amounts or concentrations of cis-element nucleic acids enable elevated expression of rep and cap genes, relative to traditional triple transfection methods. In some embodiments, reducing the amount or concentration of cis-element nucleic acids encoding cytotoxic transgenes restores the normal and necessary expression levels of rep and cap genes that are critical for rAAV production.

### Nucleic Acids

As used herein, the term “nucleic acid” refers to polymers of linked nucleotides, such as DNA, RNA, etc. In some embodiments, proteins and nucleic acids of the disclosure are isolated. In some embodiments, the DNA of a transgene is transcribed into a messenger RNA (mRNA) transcript. As used herein, the term “isolated” means artificially produced (e.g., an artificially produced nucleic acid, or an artificially produced protein, such as a capsid protein). As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been artificially produced (e.g., by chemical synthesis, by recombinant DNA technology, etc.) As used herein, a “transgene” is a nucleic acid sequence, which is not homologous to vector sequences, which encodes a polypeptide, protein, functional RNA molecule (e.g., miRNA, miRNA inhibitor) or other gene product, of interest. In some embodiments, a transgene encodes a therapeutic protein or therapeutic functional RNA. Examples of therapeutic proteins include toxins, enzymes (e.g., kinases, phosphorylases, proteases, acetylases, deacetylases, methylases, demethylases, etc.) growth factors, interleukins, interferons, anti-apoptosis factors, cytokines, anti-diabetic factors, anti-apoptosis agents, coagulation factors, anti-tumor factors, and anti-proliferative proteins. The nucleic acid coding sequence is operatively linked to regulatory components in a manner which permits transgene transcription, translation, and/or expression in a cell of a target tissue.

In some aspects, the disclosure relates to viral vectors encoding one or more transgenes that are cytotoxic or detrimental to the fitness of a host cell. A “cytotoxic” transgene refers to a transgene that encodes a gene product (e.g., a protein) that is toxic to a living cell. Examples of toxic transgenes include transgenes encoding diphtheria toxin, botulinum toxin, ribosome inactivating proteins (e.g., ricin), cytolysins, porins (e.g., actinoporins), apolipoproteins, certain proteases, etc. In some embodiments, a protein becomes cytotoxic when overexpressed in a cell. A “transgene that is detrimental to the health of a host cell” refers to a transgene encoding a protein having certain physiochemical characteristics (e.g., a secondary structure having a high thermostability, a tendency to aggregate, etc.) that results in a reduced fitness (ability to survive) of a host cell expressing that transgene relative to a host cell that does not express the transgene.

Thus, the disclosure embraces the delivery of vectors encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAPI, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18.

The nucleic acids disclosed herein may comprise a transgene to be transferred to a subject to treat a disease associated with reduced expression, lack of expression or dysfunction of the native gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco (endo) plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

The following are further non-limiting examples of proteins that may be encoded by transgenes disclosed herein to treat a disease associated with reduced expression, lack of expression or dysfunction of the native gene: a-galactosidase, acid-glucosidase, adiopokines, adiponectin, alglucosidase alfa, anti-thrombin, ApoAV, ApoCII, apolipoprotein A-I (APOA1), arylsulfatase A, arylsulfatase B, ATP-binding cassette transporter Al (ABCA1), ABCD1, CCR5 receptor, erythropoietin, Factor VIII, Factor VII, Factor IX, Factor V, fetal hemoglobin, beta-globin, GPI-anchored HDL-binding protein (GPI-HBP) I, growth hormone, hepatocyte growth factor, imiglucerase, lecithin-cholesterol acyltransferase (LCAT), leptin, LDL receptor, lipase maturation factor (LMF) 1, lipoprotein lipase, lysozyme, nicotinamide dinucleotide phosphate (NADPH) oxidase, Rab escort protein-1 (REP-1), retinal degeneration slow (RDS), retinal pigment epithelium-specific 65 (RPE65), rhodopsin, T cell receptor alpha or beta chains, thrombopocitin, tyrosine hydroxylase, VEGF, von heldebrant factor, von willebrand factor, and X-linked inhibitor of apoptosis (XIAP).

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product (e.g., a therapeutic protein or therapeutic minigene) or inhibitory RNA (e.g., shRNA, miRNA, amiRNA, miRNA inhibitor) from a transcribed gene.

### Viral Vectors

Viral vectors present a powerful tool for the delivery of plasmids and genetic material into cells. Adapting plasmid DNA for use with virus-mediated delivery has provided numerous advantages for research, including the delivery of genetic information in traditionally hard-to-transfect cells, such as neurons. Viruses naturally infect host cells and direct them to reproduce the viral genome. Scientists have taken advantage of this process by providing the virus with alternate genomes (e.g., plasmids encoding a nucleic acid or transgene), which can then be replicated once the virus has infected a host cell. In short, researchers can introduce plasmids into a host cell to generate recombinant virus.

For safety reasons, viral genomes used in research and drug development have been modified through the removal of certain genes that are required for viral replication. These genes are usually divided among numerous “accessory plasmids” which must also be present in the cell for a viral particle to be produced. The production of viral particles comprising nucleic acid(s) of interest, along with the viral genome, by a host cell is herein referred to as “packaging”. The process for the delivery and packaging of nucleic acids into viral genomes varies depending on the viral genome the nucleic acid is encoded in and will be discussed in greater detail for each viral vector below.

Recombinant adeno-associated virus (rAAV) particles are produced by introducing into a host cell, a cis-element nucleic acid comprising a transgene, a helper nucleic acid encoding adenoviral helper genes, and a packaging nucleic acid encoding Rep and/or Cap genes. A cis-element nucleic acid comprising a transgene may comprise a transgene flanked by adeno-associated virus (AAV) inverted terminal repeats (ITRs). In some embodiments, a helper nucleic acid encoding adenoviral helper genes comprises genes that mediate AAV replication (e.g., AAV E4, E2a and/or VA genes). In some embodiments, a packaging nucleic acid encodes one or more Rep genes. In some embodiments, a packaging nucleic acid encodes one or more Cap genes.

The methods of the disclosure, in some embodiments, utilize a reduced amount or concentration of cis-element nucleic acid relative to the helper nucleic acid and/or the packaging nucleic acid. In some embodiments, the amount or concentration of the cis-element nucleic acid is 10-fold to 100-fold less than the concentration of the helper nucleic acid and/or the packaging nucleic. In some embodiments, the amount or concentration of the cis-element nucleic acid is 10-fold to 100-fold, 10-fold to 90-fold, 10-fold to 80-fold, 10-fold to 70-fold, 10-fold to 60-fold, 10-fold to 50-fold, 10-fold to 40-fold, 10-fold to 30-fold, 10-fold to 20-fold, 20-fold to 100-fold, 30-fold to 100-fold, 40-fold to 100-fold, 50-fold to 100-fold, 60-fold to 100-fold, 70-fold to 100-fold, 80-fold to 100-fold, or 90-fold to 100-fold less than the concentration of the helper nucleic acid and/or the packaging nucleic acid. In some embodiments, the amount or concentration of the cis-element nucleic acid is at least 10-fold, 20-fold, 30-fold, 40-fold, 50-fold, 60-fold, 70-fold, 80-fold, 90-fold, or 100-fold less than the concentration of the helper nucleic acid and/or the packaging nucleic acid.

In some embodiments, the ratio of the concentration of amount or concentration of the cis-element nucleic acid relative to amount or concentration of the packaging nucleic acid is between 0.01:1 and 0.1:1. In some embodiments, the ratio of the concentration of amount or concentration of the cis-element nucleic acid relative to amount or concentration of the packaging nucleic acid is between 0.01:1 and 0.02:1; 0.01:1 and 0.03:1; 0.01:1 and 0.04:1; 0.01:1 and 0.05:1; 0.01:1 and 0.06:1; 0.01:1 and 0.07:1; 0.01:1 and 0.08:1; or 0.01:1 and 0.09:1. In some embodiments, the ratio of the concentration of amount or concentration of the cis-element nucleic acid relative to amount or concentration of the packaging nucleic acid is between about 0.01:1, about 0.02:1, about 0.03:1, about 0.04:1, about 0.05:1, about 0.06:1, about 0.07:1, about 0.08:1, about 0.09:1, or about 0.1:1.

In some embodiments, the ratio of the concentration of amount or concentration of the cis-element nucleic acid relative to amount or concentration of the helper nucleic acid is between 0.01:1 and 0.1:1. In some embodiments, the ratio of the concentration of amount or concentration of the cis-element nucleic acid relative to amount or concentration of the helper nucleic acid is between 0.01:1 and 0.02:1; 0.01:1 and 0.03:1; 0.01:1 and 0.04:1; 0.01:1 and 0.05:1; 0.01:1 and 0.06:1; 0.01:1 and 0.07:1; 0.01:1 and 0.08:1; or 0.01:1 and 0.09:1. In some embodiments, the ratio of the concentration of amount or concentration of the cis-element nucleic acid relative to amount or concentration of the helper nucleic acid is between about 0.01:1, about 0.02:1, about 0.03:1, about 0.04:1, about 0.05:1, about 0.06:1, about 0.07:1, about 0.08:1, about 0.09:1, or about 0.1:1.

Transgenes expressed from viral genomes for packaging in host cells can be toxic (e.g., cytotoxic or detrimental to the fitness of a host cell), and thus can interfere with viral packaging in the host cell. The present disclosure has discovered a solution to the problems of cytotoxic transgenes by reducing the amount or concentration of a cis-element nucleic acid encoding the transgene by 10-fold to 100-fold relative to other input nucleic acids (e.g., a packaging nucleic acid or a helper nucleic acid) such that the ratio of the cis-element nucleic acid relative to one of the other input nucleic acids is between 0.01:1 and 0.1:1.

As used herein, the term “recombinant virus” or “recombinant viral particle” refers to a particle produced in a host cell which encapsulates nucleic acid produced from exogenous DNA inserted into the host cell genome is, has been introduced.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells. The skilled artisan will appreciate that in methods described by the disclosure, a host cell may be transfected with 2, 3, 4, 5, 6, 7, 8, 9, 10, or more isolated nucleic acids.

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more proteins and/or one or more binding sites for inhibitory nucleic acids (e.g., shRNA, miRNAs, etc.). The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the isolated nucleic acid comprises a region (e.g., a first region) encoding an AAV2 ITR.

In some embodiments, the isolated nucleic acid further comprises one or more AAV ITRs. In some embodiments, an AAV ITR has a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, an AAV ITR is a mutant ITR (mTR) that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16 (10): 1648-1656.

As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. In some embodiments, isolated nucleic acids comprise DNA sequences encoding RNA hairpin structures (e.g. shRNA, miRNA, and amiRNA) that can serve a function similar to a mutant inverted terminal repeat (mTR) during viral genome replication, generating self-complementary AAV vector (scAAV) genomes. For example, in some embodiments, the disclosure provides rAAV (e.g. self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA (e.g., shRNA, miRNA, amiRNA, etc.). In some embodiments, the sequence encoding a hairpin-forming RNA (e.g., shRNA, miRNA, ami-RNA, etc.) is substituted at a position of the self-complementary nucleic acid normally occupied by a mutant ITR.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The instant disclosure provides a vector comprising a single, cis-acting wild-type ITR. In some embodiments, the ITR is a 5′ITR. In some embodiments, the ITR is a 3′ ITR Generally, ITR sequences are about 145 bp in length. Preferably, substantially the entire sequences encoding the ITR(s) is used in the molecule, although some degree of minor modification of these sequences is permissible. The ability to modify ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). For example, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. AAV ITR sequences may be obtained from any known AAV, including presently identified mammalian AAV types.

In some embodiments, the rAAVs of the disclosure are pseudotyped rAAVs. For example, a pseudotyped AAV vector containing the ITRs of serotype X encapsidated with the proteins of Y will be designated as AAVX/Y (e.g. AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

As described herein, the methods of producing rAAV particles involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.rh39, or AAV.rh43 serotype. In some embodiments, an AAV capsid protein is of an AAV9 serotype.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

### Adenoviral Vector

The adenovirus genome is a non-enveloped, large (36-kb) double-stranded DNA (dsDNA) molecule comprising multiple, heavily spliced transcripts. Adenoviruses have high packaging capacity (˜8 kilobases) and are able to target a broad range of dividing and non-dividing cells. Adenoviruses do not integrate into the host genome and thus only produce transient transgene expression in host cells. At either end of adenoviral genome are inverted terminal repeats (ITRs). Genes encoded by the adenoviral genome are divided into early (E1-E4) and late (L1-L5) transcripts. Most human adenoviral vectors are based on the Ad5 virus type, which uses the Coxsackie-Adenovirus Receptor to enter cells.

Recombinant adenovirus has the E1 and E3 genes deleted from its genome. Deletion of E1 renders the virus replication incompetent; E1 is supplied by adenovirus packaging cell lines, such as HEK293 cells. E3 is involved in evading host cell immunity and is thus not essential for virus production. Deletion of these two components results in a transgene packaging capacity of >8 kilobases.

Methods of the current disclosure describe recombinant adenoviral vectors encoding nucleic acid(s) of interest. Generation of recombinant adenoviral vectors involves both a transfer vector and an adenoviral vector. The transgene to be packaged in adenovirus may be initially placed in a transfer vector. Recombinant transfer vectors containing left and right flanking sequences which are complementary to the sequences at the site of insertion in the adenoviral genome facilitate insertion of the transgene into the adenoviral plasmid by homologous recombination (HR). The left and right sequences are used as templates to repair a DNA DSB in HR, thereby facilitating error-free insertion of the transgene into the adenoviral plasmid. Methods of the current disclosure describe the use of one or more accessory plasmids in a host cell. In the retroviral system, the accessory plasmid is a packaging plasmids which encodes all necessary components for viral packaging except the E1 and the E3 genes. An additional accessory plasmid is required to provide the E1 gene to the packaging cells.

### Retroviral Vector

Retrovirus (most commonly, γ-retrovirus) is an RNA virus comprised of the viral genome and several structural and enzymatic proteins, including reverse transcriptase and integrase. Once inside a host cell, the retrovirus uses the reverse transcriptase to generate a DNA provirus from the viral genome. The integrase protein then integrates this provirus into the host cell genome for production of viral genomes encoding the nucleic acid(s) of interest. Retrovirus can package relatively high amounts of DNA (up to 8 kilobases), but are unable to infect non-dividing cells and insert randomly into the host cell genome.

The retroviral transfer and packaging vectors are similar to the lentiviral system described above. Retroviral transfer vectors typically encode a nucleic acid of interest flanked by LTRs, which are derived from Moloney Murine Leukemia Virus (MoMLV) or Murine Stem Cell Virus (MSCV) sequences. Methods of the current disclosure describe the use of one or more accessory plasmids. For retroviruses, the accessory plasmids are a packaging plasmid, which encodes the gag and pol genes, and an envelope plasmid which encodes the env gene. As in the lentiviral system, the gag gene is translated into three viral core proteins: matrix (MA) proteins, which are necessary for virion assembly and infection of non-dividing cells; capsid (CA) proteins, which form the hydrophobic core of the virion; and nucleocapsid (NC) proteins, which protect the viral genome by coating and associating tightly with the RNA. The pol gene encodes for the viral protease, reverse transcriptase, and integrase enzymes which are essential for viral replication.

### Cell

A “host cell” refers to any cell that harbors, or is capable of harboring, a substance of interest or of packaging the nucleic acid of interest into a viral particle. Often a host cell is a mammalian cell. Examples of host cells include human cells, mouse cells, rat cells, dog cells, cat cells, hamster cells, monkey cells, insect cells, plant cells, or bacterial cells. Examples of insect cells include but are not limited to Spodoptera frugiperda (e.g., Sf9, Sf21), Spodoptera exigua, Heliothis virescens, Helicoverpa zea, Heliothis subflexa, Anticarsia gemmatalis, Trichopulsia ni (e.g., High-Five cells), Drosophila melanogaster (e.g., S2, S3), Antheraea eucalypti, Bombyx mori, Aedes alpopictus, Aedes aegyptii, and others. Examples of bacterial cells include, but are not limited to Escherichia coli, Corynebacterium glutamicum, and Pseudomonas fluorescens. Examples of yeast cells include but are not limited to Saccharomyces cerevisiae, Saccharomyces pombe, Pichia pastoris, Bacillus sp., Aspergillus sp., Trichoderma sp., and Myceliophthora thermophila C1. Examples of plant cells include but are not limited to Nicotiana sp., Arabidopsis thaliana, Mays zea, Solanum sp., or Lemna sp.

In some embodiments, a host cell is a mammalian cell. Examples of mammalian cells include Henrietta Lacks tumor (HeLa) cells and baby hamster kidney (BHK-21) cells. In some embodiments, a host cell is a human cell, for example a HEK293T cell. A host cell may be used as a recipient of one or more viral transfer vectors and one or more accessory plasmids. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

### EXAMPLES

### Example 1. DEVELOPMENT OF LOW-CIS TRIPLE TRANSFECTION METHOD THAT GENERATES HIGH-QUALITY AAV vectors with significantly reduced plasmid demand

FIGS. 1A-1B demonstrate the benefits of utilizing the low-cis triple transfection methods disclosed herein over traditional triple transfection methods. In standard triple transfection (FIG. 1A, left), three plasmids are co-transfected to cells at roughly equal molar ratio: a helper plasmid that delivers certain genes of adenovirus (pAd-Helper), a trans plasmid that expresses AAV rep and cap genes (pTrans), and a cis plasmid that harbors a therapeutic transgene cassette flanked by AAV inverted terminal repeats (pCis). The proposed low-cis triple transfection (FIG. 1A, right) reduces pCis amount to 10% or 1% of the standard amount. This method effectively packages rAAVs encoding cytotoxic transgene that is not compatible with standard triple transfection. In addition, it generates comparable or slightly reduced titer of rAAV encoding non-toxic transgene but saves more than 90% of cis-plasmid cost. Importantly, the quality of rAAV generated by low-cis triple transfection is superior in terms of vector purity and in vivo tissue transduction efficiency. FIG. 1B summarizes how low-cis triple transfection overcomes several limitations of standard triple transfection in rAAV manufacturing.

Methods of the disclosure that utilized reduced concentrations of cis-element nucleic acid produced titers of rAAV encoding non-toxic transgene in HEK293 cells that were comparable to traditional triple transfection methods. HEK293 cells were transfected with varying amounts of cis-element plasmid, a fixed amount of helper plasmid and a fixed amount of packaging plasmid. The cis-element plasmid comprised self-complementary AAV9 and a transgene encoding enhanced green fluorescent protein (eGFP) with a CB6 promoter (CMV enhancer, chicken β-actin promoter and hybrid intron) and flanked by AAV9 ITRs. Following the transfection, rAAV particles were purified by CsCl gradient centrifugation from 1×10⁹ transfected HEK293 cells. rAAV titer (genome copy (GC)/mL) was determined by ddPCR.

Three different experimental conditions were tested to vary the amount of the cis-element plasmid, as shown in Table 1.

TABLE 1

| | Relative amount | | Relative amount | |
|---|---|---|---|---|
| Experimental | of cis-element | | of packaging | Relative amount of |
| Group | plasmid | | plasmid | helper plasmid |
| 1 | 1 | (100%) | 1 (100%) | 1 (100%) |
| 2 | 0.1 | (10%) | 1 (100%) | 1 (100%) |
| 3 | 0.01 | (1%) | 1 (100%) | 1 (100%) |

It was found that reducing the amount of the cis-element plasmid by 10-fold (10%) or 100-fold (1%) generated comparable rAAV9 yield in HEK293 cells, compared to the traditional triple transfection ratio (100% cis-element plasmid input) (FIG. 1C). These data demonstrate that reducing the amount of cis-element nucleic acid does not negatively impact rAAV titers.

### Example 2. Low-Cis Triple Transfection Generates Robust Titer of rAAV Encoding Cytotoxic Transgenes, which is Incompatible with Standard Triple Transfection Methods

Methods of the disclosure that utilized reduced concentrations of cis-element nucleic acid produced titers of rAAV encoding non-toxic transgene in HEK293 cells generated robust titers of rAAV encoding cytotoxic transgenes that exceeded titers generated using traditional methods.

HEK293 cells were transfected with varying amounts of cis-element plasmid, a fixed amount of helper plasmid and a fixed amount of packaging plasmid. The cis-element plasmid comprised either (a) AAV PHP.eB vector with AAV PHP.eB ITRs and a transgene encoding suppressor tRNA (stRNA19 or stRNA21) (known to be cytotoxic transgenes); or (b) self-complementary AAV9 and a transgene encoding human FOXG1 (wild-type or optimized) (known to be cytotoxic transgenes) with a CB6 promoter (CMV enhancer, chicken β-actin promoter and hybrid intron) and flanked by AAV9 ITRs. Following the transfection, rAAV particles were purified by CsCl gradient centrifugation from 1×10⁹ transfected HEK293 cells. rAAV titer (genome copy (GC)/mL) was determined by ddPCR.

Experimental conditions for expression of the suppressor tRNAs were tested to vary the amount of the cis-element plasmid, as shown in Table 2.

TABLE 2

| | | | | Relative | |
|---|---|---|---|---|---|
| | Relative amount | | | amount of | Relative |
| Experimental | of cis-element | | | packaging | amount of |
| Group | plasmid | | Transgene | plasmid | helper plasmid |
| 1 | 1 | (100%) | stRNA19 | 1 (100%) | 1 (100%) |
| 2 | 0.1 | (10%) | stRNA19 | 1 (100%) | 1 (100%) |
| 3 | 0.01 | (1%) | stRNA19 | 1 (100%) | 1 (100%) |
| 4 | 1 | (100%) | stRNA21 | 1 (100%) | 1 (100%) |
| 5 | 0.1 | (10%) | stRNA21 | 1 (100%) | 1 (100%) |
| 6 | 0.01 | (1%) | stRNA21 | 1 (100%) | 1 (100%) |

Experimental conditions for expression of the FOXG1 genes were tested to vary the amount of the cis-element plasmid, as shown in Table 3.

TABLE 3

| | Relative | | | Relative | Relative |
|---|---|---|---|---|---|
| | amount of cis- | | | amount of | amount of |
| Experimental | element | | | packaging | helper |
| Group | plasmid | | Transgene | plasmid | plasmid |
| 1 | 1 | (100%) | Human FOXG1 | 1 (100%) | 1 (100%) |
| 2 | 0.1 | (10%) | Human FOXG1 | 1 (100%) | 1 (100%) |
| 3 | 0.01 | (1%) | Human FOXG1 | 1 (100%) | 1 (100%) |
| 4 | 1 | (100%) | Codon-optimized | 1 (100%) | 1 (100%) |
| | | | FOXG1 | | |
| 5 | 0.1 | (10%) | Codon-optimized | 1 (100%) | 1 (100%) |
| | | | FOXG1 | | |
| 6 | 0.01 | (1%) | Codon-optimized | 1 (100%) | 1 (100%) |
| | | | FOXG1 | | |

It was found that reducing the amount of the cis-element plasmid by 10-fold (10%) or 100-fold (1%) generated significant increases in rAAV yield in HEK293 cells, compared to the traditional triple transfection ratio (100% cis-element plasmid input), for expression of the suppressor tRNAs (FIG. 2A) and the FOXG1 genes (FIG. 2B). In the case of FOXG1, there was no expression of rAAV in the method that utilized the traditional triple transfection ratio. Furthermore, reducing the amount of the cis-element plasmid (that encoded the suppressor tRNAs) improved the expression levels of Rep and Cap (VP1, VP2, VP3) proteins as revealed by Western blot, relative to the traditional triple transfection ratio (FIG. 2C). These data demonstrate that reducing the amount of cis-element nucleic acid enables surprising and robust expression of rAA Vs encoding cytotoxic transgenes.

To further examine the effect of utilizing the methods of the disclosure to package cytotoxic transgenes, rAAVs were generated encoding mCherry-tagged cytotoxic UGA suppressor tRNAs (sup-tRNA19 and sup-tRNA21) using cis-element plasmids at 100%, 10%, or 1% relative to the amount of packaging and helper plasmids. In a standard triple transfection, the yield of rAAV encoding the cytotoxic UGA suppressor tRNAs sup-tRNA19 and sup-tRNA21 is less than 5% of non-toxic control (AAV9.mCherry) (FIG. 3A, compare lanes 2 and 5 to lane 1). However, AAV9.mCherry.sup-tRNA19 and 21 yield was increased more than 10-fold when reducing the cis-element amount to 10% or 1%, reaching more than 50% of non-toxic control (FIG. 3A, compare lanes 3, 4, 6 and 7 to lane 1). rAAV was harvested from HEK293 cell crude lysate in 12-well plate production scale. DNAse-resistant genome copy per mL crude lysate (GC/mL) was determined by ddPCR.

To then test the functionality of the rAAVs encoding cytotoxic tRNAs, HEK293 cells expressing EGFP and Gluc with a premature stop codon were infected with AAV9.mCherry.sup-tRNA19 or 21 generated using 100%, 10% or 1% of cis-element plasmids as shown in FIG. 3A. Through this method, HEK293 cells expressing the cytotoxic tRNAs would read through the premature stop codon to express full-length EGFP and Gluc, and show fluorescence and Gluc activity. One-fifth of the crude lysate was added to HEK293 cells transfected with EGFP and Gluc reporter plasmid and infected with wide-type Adenovirus 5 with MOI of 100. Images and measurements were taken at Day 2 post transfection. FIGS. 3B-3D demonstrate that the UGA suppressor tRNA packaged by low-cis triple transfection is functional in rescuing EGFP and Gluc premature UGA stop codon. FIG. 3E demonstrates that compared with standard triple transfection, dramatic reduction of sup-tRNA pCis amount rescues the expression levels of Rep (Rep68, Rep52 and Rep40) and Cap (VP1, VP2, VP3) proteins to normal levels. GAPDH is the internal control of Western blot. Data are mean+s.d. of three biological replicates. * p<0.05, ** p<0.01, *** p<0.001. ns, not significant.

### Example 3. Low-Cis Triple Transfection for Packaging of Non-Toxic Transgenes

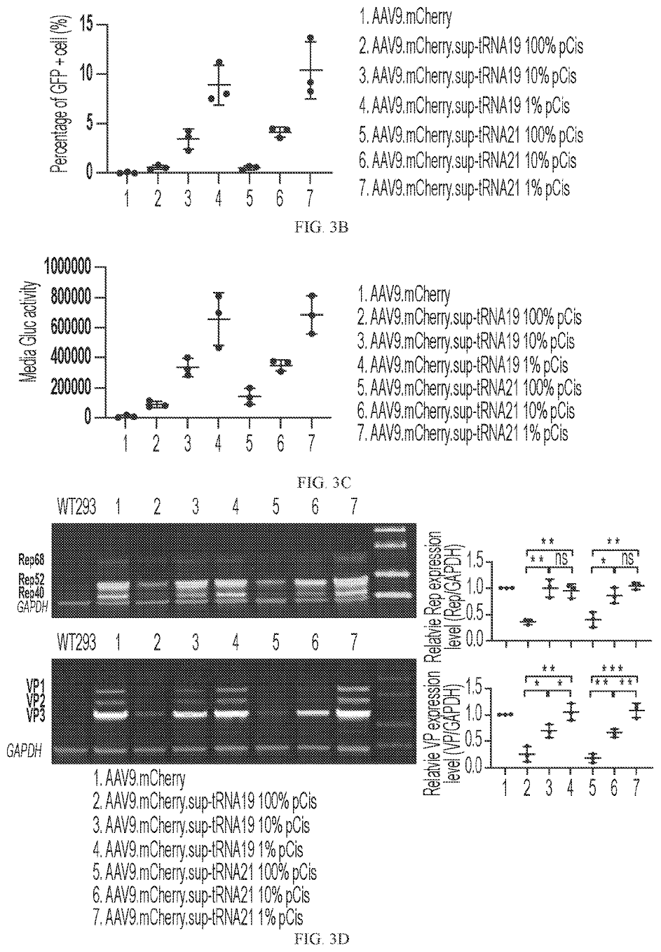

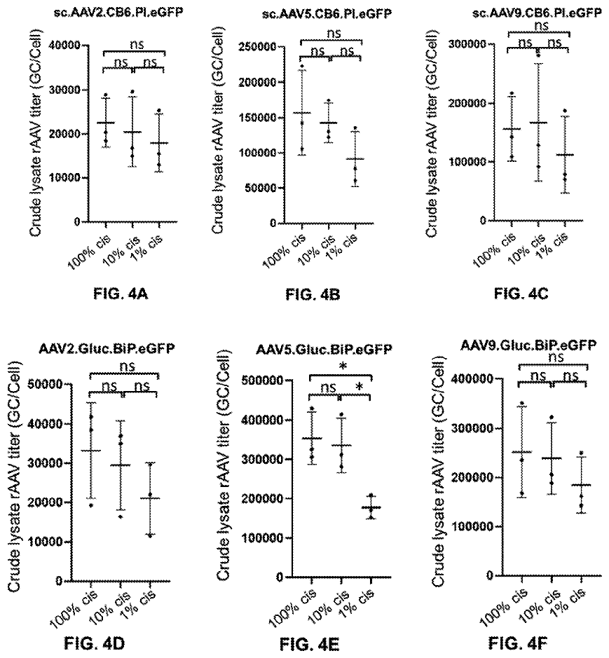

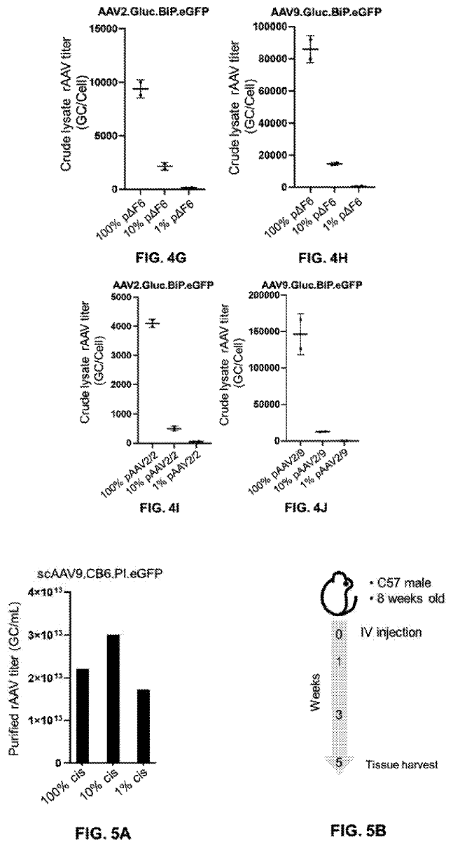

The low-cis transfection method disclosed herein showed compatibility with multiple AAV serotypes and transgenes. A self-complementary (sc) transgene expressing EGFP (CB6.PI.cGFP) was packaged into rAAV serotypes 2, 5 and 9. FIGS. 4A-4C demonstrate that the low-cis triple transfection method produces rAAV titers comparable to traditional triple transfection methods across all serotypes tested. Similarly, sc transgene expressing Gluc and eGFP under a bi-directional promoter (Gluc.BiP.eGFP) was packaged into rAAV serotypes 2, 5 and 9. FIGS. 4D-4F demonstrate that the low-cis triple transfection method produces rAAV titers comparable to traditional triple transfection methods across all serotypes tested. Data are mean+s.d. of three biological replicates. * p<0.05, ns, not significant.

FIGS. 4G-4J show that decreasing the input levels of pAd-helper or pTrans plasmids lead to an almost linear drop in production yield of AAV2 and AAV9 vectors. These data demonstrate that while significantly decreasing cis-element plasmid levels is compatible with maintenance of rAAV production, low levels of pAd-helper or pTrans plasmids are not equally compatible. Data are mean+s.d. of two biological replicates.

### Example 4. Low-Cis Triple Transfection Robustly Packages scAAV9.CB6.Pl.eGFP at a Large Scale with Superior Tissue Transduction Efficiency

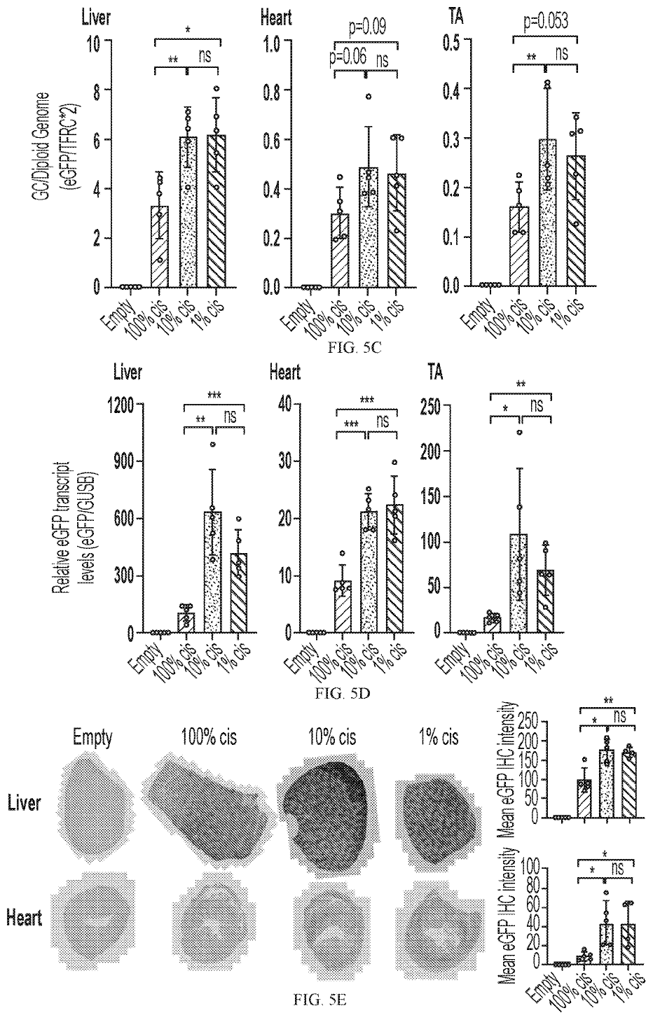

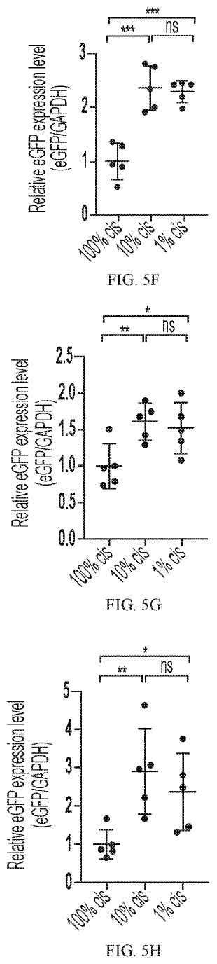

The transduction efficiency of low-cis triple transfection-generated rAAV was tested in vivo. A cis-element plasmid encoding scAAV9.CB6.PI.cGFP was used in different amounts (100%, 10% or 1%) to generate self-complementary rAAV9 particles. The rAAV9 particles were purified by CsCl gradient centrifugation from 1E9 transfected HEK293 cells, and the titer was determined by digital droplet PCR (ddPCR) (reported in genome copy (GC)/mL). FIG. 5A demonstrates the titer of the rAAV9 generated using 100%, 10% or 1% cis-element. Compared to a standard triple transfection (i.e., 100% cis-element input), reducing the cis-element encoding scAAV9.CB6.PI.cGFP by 10-fold or 100-fold generates comparable rAAV9 yield in HEK293 cells. FIG. 5B depicts the working scheme of assaying the in vivo transduction efficiency of scAAV9.CB6.PI.eGFP. C57 male mice were dosed with 5E11 GC per mouse, and a control group was dosed with empty capsid. Tissues (liver, heart and tibialis anterior [TA]) were extracted and analyzed for eGFP DNA, RNA and protein expression 5 weeks following transduction. DNA was quantified by ddPCR with Taqman probes against either eGFP transgene or internal control Tfre gene. rAAV9 generated by low-cis triple transfection shows superior transduction at DNA levels in assayed tissues (FIG. 5C). To examine RNA expression, extracted tissue RNA was converted to cDNA, then quantified by ddPCR with Taqman probes against either eGFP transgene or internal control Gusb gene. rAAV generated by low-cis triple transfection showed superior mRNA expression levels in assayed tissues (FIG. 5D). Immunohistochemical analyses were performed for eGFP expression in liver and hearts from transduced animals. Representative IHC images showing the eGFP protein expression levels in liver and hearts from mice transduced with differently generated rAAV9 are shown in FIG. 5E (left). Quantification of IHC signal intensity is shown in FIG. 5E, right. FIGS. 5F-5H show Western blot images (left) and quantification (right) of eGFP expression levels from transduction of scAAV9.CB6.PI.cGFP packaged by 100%, 10% or 1% pCis input in mouse liver (FIG. 5F), heart (FIG. 5G) and TA (FIG. 5H). GAPDH serves as the internal control. Data are mean+s.d. of individual animals. * p<0.05, ** p<0.01, *** p<0.001. ns, not significant.

### Example 5. Low-Cis Triple Transfection is Effective in Packaging a Variety of Transgenes and Serotypes

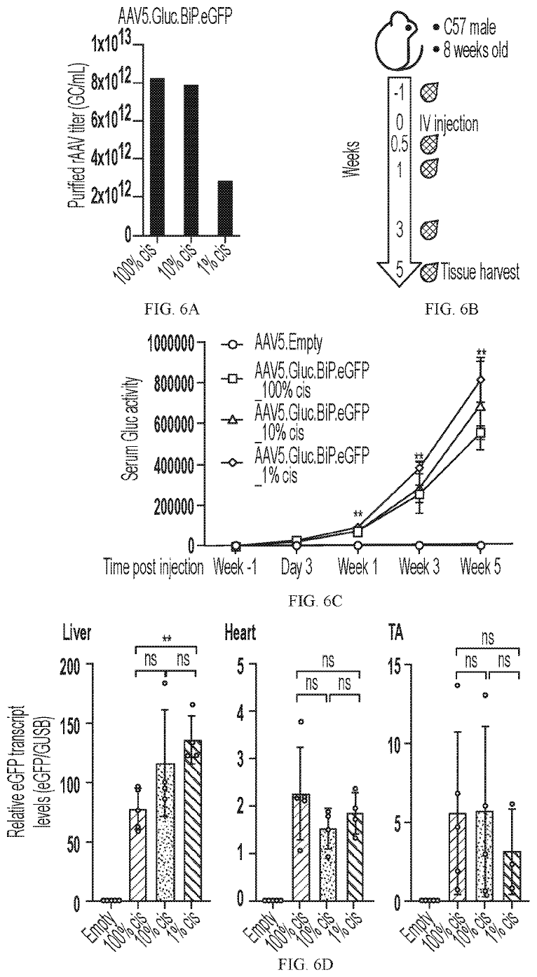

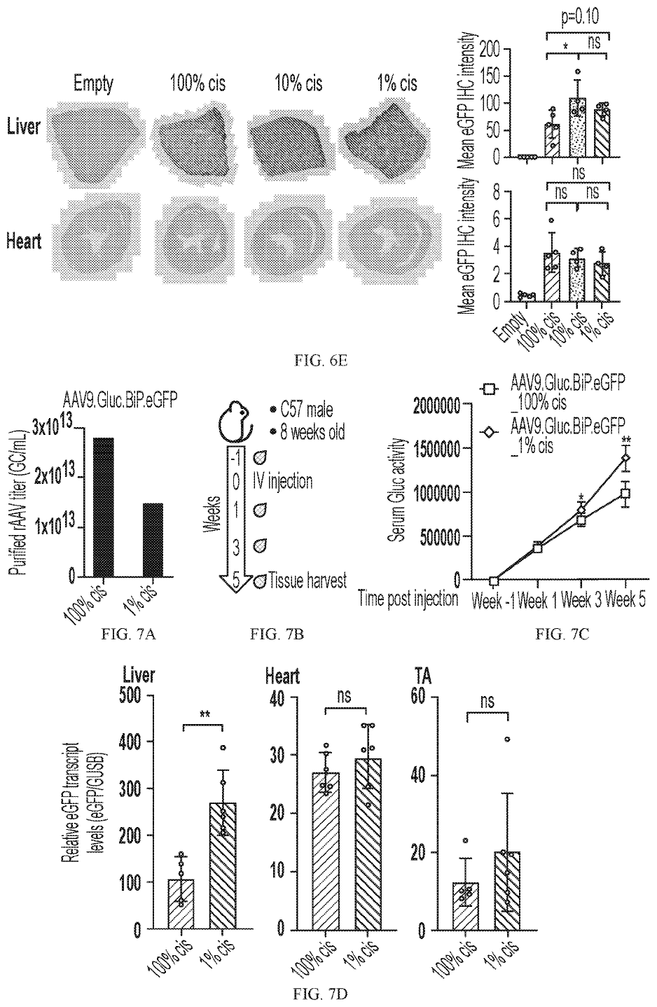

The transduction efficiency of low-cis triple transfection-generated rAAV was tested in vivo. A cis-element plasmid encoding AAV5.Gluc.BiP.eGFP was used in different amounts (100%, 10% or 1%) to generate rAAV5 particles. The rAAV5 particles were purified by CsCl gradient centrifugation from 1E9 transfected HEK293 cells, and the titer was determined by digital droplet PCR (ddPCR) (reported in genome copy (GC)/mL). FIG. 6A demonstrates the titer of the rAAV5 generated using 100%, 10% or 1% cis-element. FIG. 6B depicts the working scheme of assaying the in vivo transduction efficiency of rAAV5.Gluc.BiP.eGFP. C57 male mice were dosed with 5E11 GC per mouse, and a control group was dosed with empty capsid. Blood was collected 1 week prior to IV injection of rAAV5, then at time points of 3 days, 1 week, 3 weeks and 5 weeks. Tissues (liver, heart and tibialis anterior [TA]) were extracted and analyzed for eGFP RNA and protein expression 5 weeks following transduction. Gluc activity in the blood was measured and showed enhanced activity when using rAAV5 packaged with 1% cis-element compared to 100% cis-element and empty capsid (FIG. 6C). Shown are mean+s.d., * denotes comparison between 100% cis and 1% cis group. To examine RNA expression, extracted tissue RNA was converted to cDNA, then quantified by ddPCR with Taqman probes against either eGFP transgene or internal control Gusb gene. rAAV5 generated by low-cis triple transfection showed superior mRNA expression levels in liver but not heart or muscle tissue (FIG. 6D). Immunohistochemical analyses were performed for eGFP expression in liver and hearts from transduced animals. Representative IHC images showing the eGFP protein expression levels in liver and hearts from mice transduced with differently generated rAAV5 are shown in FIG. 6E (left). Quantification of IHC signal intensity is shown in FIG. 6E, right. Data are mean+s.d. of individual animals. * p<0.05, ** p<0.01, ns, not significant.

### Example 6. Low-Cis Triple Transfection is Effective in Packaging Single-Stranded Transgenes

The transduction efficiency of low-cis triple transfection-generated rAAV was tested in vivo. A cis-element plasmid encoding AAV9.Gluc.BiP.cGFP was used in different amounts (100% or 1%) to generate rAAV9 particles. The rAAV9 particles were purified by CsCl gradient centrifugation from 1E9 transfected HEK293 cells, and the titer was determined by digital droplet PCR (ddPCR) (reported in genome copy (GC)/mL). FIG. 7A demonstrates the titer of the rAAV9 generated using 100% or 1% cis-element. FIG. 7B depicts the working scheme of assaying the in vivo transduction efficiency of rAAV9.Gluc.BiP.eGFP. C57 male mice were dosed with 5E11 GC per mouse, and a control group was dosed with empty capsid. Blood was collected 1 week prior to IV injection of rAAV9, then at time points of 1 week, 3 weeks and 5 weeks. Tissues (liver, heart and tibialis anterior [TA]) were extracted and analyzed for eGFP RNA and protein expression 5 weeks following transduction. Gluc activity in the blood was measured and showed enhanced activity when using rAAV9 packaged with 1% cis-element compared to 100% cis-element and empty capsid (FIG. 7C). Shown are mean+s.d., * denotes comparison between 100% cis and 1% cis group. To examine RNA expression, extracted tissue RNA was converted to cDNA, then quantified by ddPCR with Taqman probes against either eGFP transgene or internal control Gusb gene. rAAV9 generated by low-cis triple transfection showed superior mRNA levels in liver but not heart or muscle (TA) tissue (FIG. 7D). To measure function of the delivered protein, GFP fluorescence was measured in protein lysate from livers collected from the mice transduced with rAAV9. While GFP signal was undetected in heart and muscle tissues likely due to low transduction efficiency, the liver tissue samples showed an increase in GFP fluorescence from the 1% cis group compared to the 100% cis group, indicating greater protein expression in the 1% cis group. FIG. 7F shows Western blot images (left) and quantification (right) of eGFP expression levels from transduction of rAAV9.Gluc.BiP.eGFP packaged by 100% or 1% pCis input in mouse liver, heart and TA (FIG. 5H). GAPDH serves as the internal control. Data are mean+s.d. of individual animals. * p<0.05, ** p<0.01, *p<0.001. ns, not significant. The liver-specific superior transduction of low-cis triple transfection-generated rAAV may be caused by change of serotype (e.g., AAV5 vs. AAV9) or transgene (e.g., single-stranded vs. self-complementary). Considering that a similar phenomenon was observed by single-stranded rAAV9.Gluc.BiP.eGFP in Example 5, these data suggest that vector genome configuration plays an important role in tissue transduction efficiency.

### Example 7. Low-Cis Triple Transfection Generation of rAAV Confers Enhanced Vector Purity

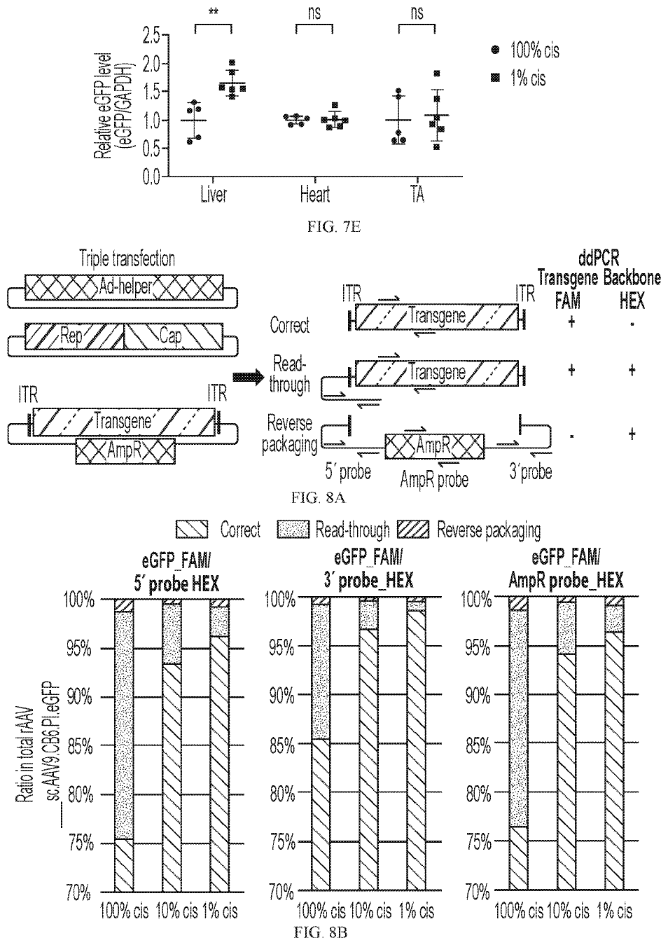

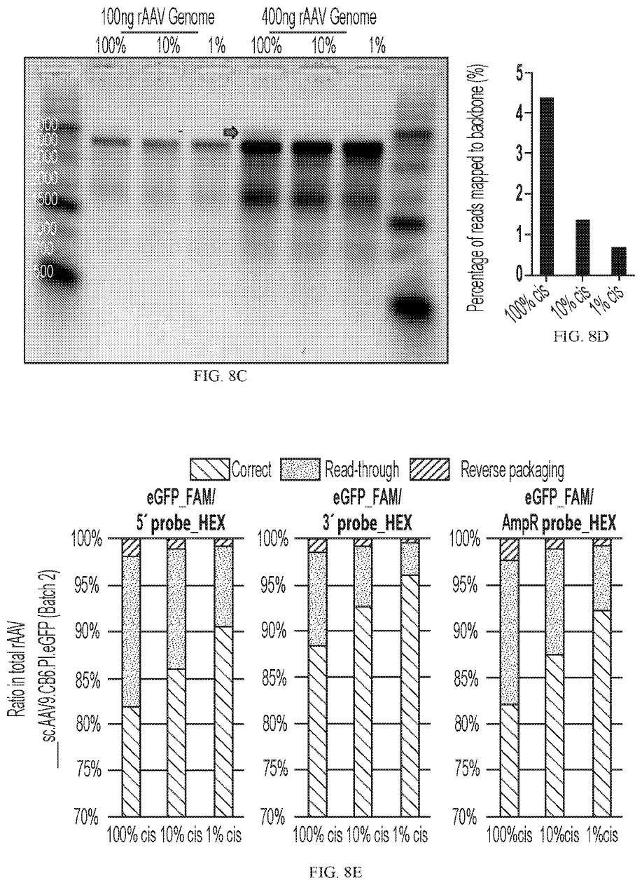

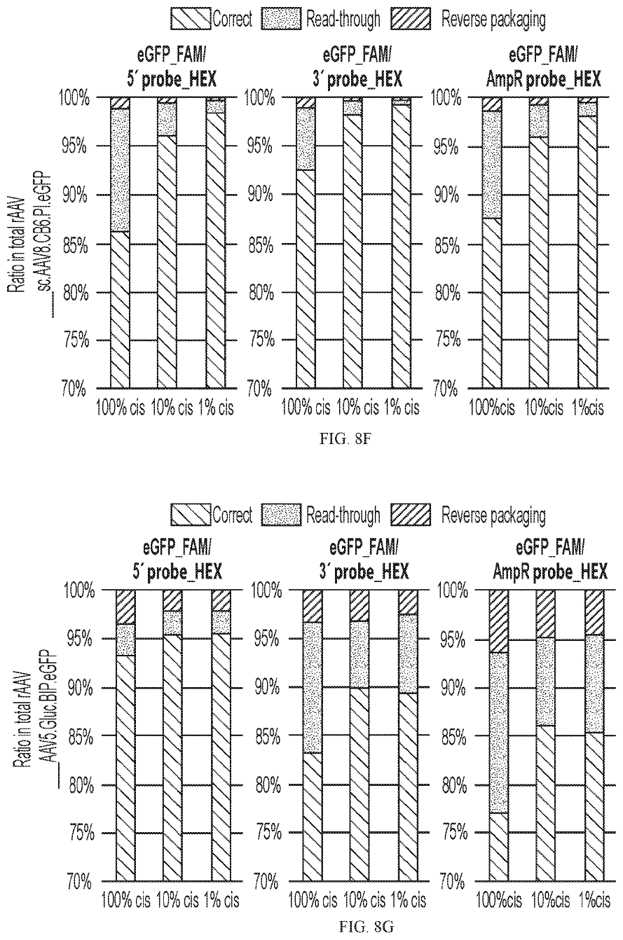

Traditionally, two major sources of AAV vector impurity are: (1) read-through rAAV, which packages both the transgene and the plasmid backbone; and (2) reverse-packaged rAAV, which packages the plasmid backbone. A ddPCR assay was designed to detect rAAV with read-through and reverse packaging by designing Taqman probes against the transgene or plasmid backbone (5′, 3′, or Ampicillin-resistant gene (AmpR)) with different dyes. This method allowed for distinguishing accurate read-through and reverse-packaging rAAVs, as shown in the schematic of FIG. 8A. rAAVs were purified by CsCl gradient centrifugation from 1E9 transfected HEK293 cells and the DNA analyzed. FIG. 8B demonstrates that rAAV produced by low-cis triple transfection shows much less read-through or reverse-packaged rAAV compared to traditional triple transfection. FIG. 8C shows a denaturing alkaline gel, wherein the oversized read-through rAAV band (indicated by the arrow) is diminished when the pCis input is reduced to 10% or 1%. Furthermore, PacBio sequencing showed the reads mapped to plasmid backbone, as indicated by the arrow, were significantly decreased with 10% or 1% pCis input (FIG. 8D). The same attenuation of read-though and reverse-packaged rAAV was observed with different batches, serotypes, and transgenes by low-cis triple transfection (FIGS. 8E-8G).

These data demonstrate that the low-cis transfection methods of the disclosure produce rAAV with higher purity than conventional transfection methods. Specifically, these data show that low-cis transfection methods of the disclosure produce rAAV with greater than 95% purity (i.e., less than 5% read-through and reverse packaging rAAV).

What is claimed is:

1. A method for producing recombinant adeno-associated virus (rAAV), the method comprising introducing into a host cell:
(i) a cis-element nucleic acid comprising a transgene;
(ii) a helper nucleic acid encoding adenoviral helper genes; and
(iii) a packaging nucleic acid encoding Rep and/or Cap genes;
wherein the concentration of the cis-element nucleic acid that is introduced to the host cell is 10-fold to 100-fold less than the concentration of the helper nucleic acid and/or the packaging nucleic acid that is introduced to the host cell.

2. A method for producing recombinant adeno-associated virus (rAAV), the method comprising introducing into a host cell:
(i) a specific concentration of cis-element nucleic acid comprising a transgene;
(ii) a specific concentration of helper nucleic acid encoding adenoviral helper genes; and
(iii) a specific concentration of packaging nucleic acid encoding Rep and/or Cap genes;
wherein the ratio of (i): (ii) and/or the ratio of (i): (iii) is between 0.01:1 and 0.1:1.

3. The method of claim 1 or 2, wherein the host cell is a viral vector packaging cell.

4. The method of any one of the preceding claim, wherein the host cell is a mammalian cell.

5. The method of any one of the preceding claim, wherein the host cell is a human cell, optionally a HEK 293T cell.

6. The method of any one of claims 1-3, wherein the host cell is an insect cell, optionally a Spodoptera frugiperda (Sf9) cell.

7. The method of any one of the preceding claims, wherein the cis-element nucleic acid, the helper nucleic acid, and/or the packaging nucleic acid is a plasmid.

8. The method of any one of the preceding claims, wherein the cis-element nucleic acid comprises a transgene flanked by two inverted terminal repeats (ITRs).

9. The method of claim 8, wherein at least one of the ITRs is an AAV1, AAV2, AAV3, AAV4, AAV5, AAV6, AAV7, AAV8, or AAV9 ITR.

10. The method of any one of the preceding claims, wherein the cis-element nucleic acid is a self-complementary nucleic acid comprising at least one AITR or mTR.

11. The method of any one of the preceding claims, wherein the transgene encodes a protein, optionally a therapeutic protein.

12. The method of any one of the preceding claims, wherein the transgene is cytotoxic or comprises one or more physiochemical characteristics that are detrimental to the fitness of the host cell (e.g., the transgene encodes a protein that forms a secondary structure with high thermal stabilities).

13. The method of any one of the preceding claims, wherein the adenoviral helper genes comprise E4, E2a and/or VA genes.

14. The method of any one of the preceding claims, wherein the concentration of the cis-element nucleic acid that is introduced to the host cell is 10-fold to 100-fold, 10-fold to 90-fold, 10-fold to 80-fold, 10-fold to 70-fold, 10-fold to 60-fold, 10-fold to 50-fold, 10-fold to 40-fold, 10-fold to 30-fold, or 10-fold to 20-fold less than the concentration of the helper nucleic acid and/or the packaging nucleic acid that is introduced to the host cell.

15. The method of any one of the preceding claims, wherein the ratio of the concentration of (i) relative to (ii) is between 0.01:1 and 0.1:1, optionally between 0.01:1 and 0.05:1 or between 0.01:1 and 0.025:1.

16. The method of any one of the preceding claims, wherein the ratio of the concentration of (i) relative to (iii) is between 0.01:1 and 0.1:1, optionally between 0.01:1 and 0.05:1 or between 0.01:1 and 0.025:1.

17. The method of any one of the preceding claims, wherein the cis-element nucleic acid, the helper nucleic acid, and/or the packaging nucleic acid are introduced into the host cell simultaneously.

18. The method of any one of the preceding claims, wherein a composition comprising the cis-element nucleic acid, the helper nucleic acid, and/or the packaging nucleic acid is introduced into the host cell.

19. The method of any one of claims 1-16, wherein the cis-element nucleic acid, the helper nucleic acid, and/or the packaging nucleic acid are introduced into the host cell separately.

20. The method of any one of the preceding claims, wherein the helper nucleic acid, and/or the packaging nucleic acid are introduced into the host cell using electroporation or transfection techniques.

21. The method of any one of the preceding claims, wherein less than 5% of the produced rAAV produced comprise read-through rAAV or reverse packaging rAAV.

22. The method of any one of the preceding claims, wherein the method produces a lower relative amount of read-through rAAV or reverse packaging rAAV compared to a traditional triple-transfection method, optionally wherein the traditional triple-transfection method produces rAAVs using a 1:1 ratio for (i): (ii) and/or (i): (iii).

23. The method of claim 21 or 22, wherein the amount of read-through rAAV and reverse packaging rAAV is determined by digital droplet PCR.

24. A composition comprising:
(i) a cis-element nucleic acid comprising a transgene;
(ii) a helper nucleic acid encoding adenoviral helper genes; and
(iii) a packaging nucleic acid encoding Rep and/or Cap genes;
wherein the ratio of (i): (ii) and/or the ratio of (i): (iii) is between 0.01:1 and 0.1:1.

25. The composition of claim 20, wherein the cis-element nucleic acid, the helper nucleic acid, and/or the packaging nucleic acid is a plasmid.

26. The composition of claim 20 or 21, wherein the cis-element nucleic acid comprises a transgene flanked by two inverted terminal repeats (ITRs).

27. The composition of claim 22, wherein at least one of the ITRs is an AAV1, AAV2, AAV3, AAV4, AAV5, AAV6, AAV7, AAV8, or AAV9 ITR.

28. The composition of any one of claims 20-23, wherein the cis-element nucleic acid is a self-complementary nucleic acid comprising at least one AITR or mTR.

29. The composition of any one of claims 20-24, wherein the transgene encodes a protein, optionally a therapeutic protein.

30. The composition of any one of claims 20-25, wherein the transgene is cytotoxic or comprises one or more physiochemical characteristics that are detrimental to the fitness of the host cell (e.g., the transgene encodes a protein that forms a secondary structure with high thermal stabilities).

31. The composition of any one of claims 20-26, wherein the adenoviral helper genes comprise E4, E2a and/or VA genes.

32. The composition of any one of claims 20-27, wherein the concentration of the cis-element nucleic acid is 10-fold to 100-fold, 10-fold to 90-fold, 10-fold to 80-fold, 10-fold to 70-fold, 10-fold to 60-fold, 10-fold to 50-fold, 10-fold to 40-fold, 10-fold to 30-fold, or 10-fold to 20-fold less than the concentration of the helper nucleic acid and/or the packaging nucleic.

33. The composition of any one of claims 20-28, wherein the ratio of the concentration of (i) relative to (ii) is between 0.01:1 and 0.1:1, optionally between 0.01:1 and 0.05:1 or between 0.01:1 and 0.025:1.

34. The composition of any one of claims 20-29, wherein the ratio of the concentration of (i) relative to (iii) is between 0.01:1 and 0.1:1, optionally between 0.01:1 and 0.05:1 or between 0.01:1 and 0.025:1.
